(19) United States
(12) Patent Application Publication
(10) Pub. No.: US 2024/0189456 A1
(43) Pub. Date: Jun. 13, 2024

(54) Treatment Of Prion Diseases

(71) Applicant: Regeneron Pharmaceuticals, Inc., Tarrytown, NY (US)

(72) Inventors: Sahar Gelfman, Tarrytown, NY (US); Andreja Avbersek, Tarrytown, NY (US); Sarah Wolf, Tarrytown, NY (US); Alicia Hawes, Tarrytown, NY (US); Giovanni Coppola, Tarrytown, NY (US); Vijay Kumar Manickavasagam Pounraja, Tarrytown, NY (US)

(21) Appl. No.: 18/535,105

(22) Filed: Dec. 11, 2023

Related U.S. Application Data
(60) Provisional application No. 63/431,908, filed on Dec. 12, 2022.

(51) Int. Cl. A61K 48/00; A61K 38/00; C12N 15/861

(52) U.S. Cl. CPC A61K 48/005; A61K 38/00; C12N 15/861

(57) ABSTRACT

Methods of treating subjects having a prion disease or at risk of developing a prion disease by administering a nucleic acid molecule encoding a modified Prion protein (PrP), and methods of identifying a subject that is a candidate for treatment or prevention of a prion disease are presented herein.

### REFERENCE TO SEQUENCE LISTING

This application includes a Sequence Listing filed electronically as an XML file named 381203969SEQ, created on December 6, with a size of 4,484 bytes. The Sequence Listing is incorporated herein by reference.

### FIELD

The present disclosure generally relates to the treatment of subjects having a prion disease or at risk of developing a prion disease, by administering a nucleic acid molecule encoding a modified Prion protein (PrP), and methods of identifying a subject that is a candidate for treatment or prevention of a prion disease.

### BACKGROUND

Prion diseases (neurodegenerative spongiform encephalopathies) are caused by the conversion of the natively folded prion protein (PrP) into self-propagating and misfolded scrapie PrP that results in scrapie PrP aggregation, the accumulation of scrapie PrPSc deposits, synaptic damage and dendrite loss, spongiform degeneration, and brain inflammation leading to neuronal death and inherited prion diseases. In some instances, particular prion gene (PRNP) mutations can cause this misfolding of the PrP protein. Symptoms include: behavioral changes, gait ataxia, myoclonus, cognitive difficulties and rapidly progressive dementia, and various visual impairments. Octapeptide repeats within PrP promote prion aggregation and deletion of octapeptide repeats reduce prion aggregation is dose dependent (Yu et al., Int. J. Mol. Sci., 2021, 22, 1800). There are no existing therapies for prion disease.

### SUMMARY

The present disclosure provides methods of treating a subject having a prion disease or at risk of developing a prion disease, the methods comprising: a) administering a nucleic acid molecule encoding a modified PrP to the subject; or b) administering an inhibitory nucleic acid molecule that targets a region of a PRNP that comprises a genetic variation that causes production of a scrapie PrP; wherein the subject comprises at least one reference PRNP allele or comprises at least one PRNP allele encoding an endogenous scrapie PrP; wherein the nucleic acid molecule encoding the modified PrP administered to the subject comprises a nucleotide sequence encoding PrP but having a deletion that comprises 24 nucleotides within the nucleotide sequence encoding the octapeptide repeat region.

The present disclosure also provides methods of treating a subject having a prion disease or at risk of developing a prion disease by administering a nucleic acid molecule encoding a modified PrP to the subject, the methods comprising: determining or having determined whether the subject comprises at least one reference PRNP allele or comprises at least one PRNP allele encoding an endogenous scrapie PrP, by: obtaining or having obtained a biological sample from the subject; and performing or having performed a sequence analysis on the biological sample to determine if the subject has a genotype comprising at least one reference PRNP allele or comprising at least one PRNP allele encoding an endogenous scrapie PrP; and administering or continuing to administer the nucleic acid molecule encoding the modified PrP to the subject that has at least one reference PRNP allele or has at least one PRNP allele encoding an endogenous scrapie PrP; wherein the nucleic acid molecule encoding the modified PrP administered to the subject comprises a nucleotide sequence encoding PrP but having a deletion that comprises 24 nucleotides within the nucleotide sequence encoding the octapeptide repeat region; and wherein the presence of at least one reference PRNP allele or at least one PRNP allele encoding an endogenous scrapie PrP indicates the subject has an increased risk of developing a prion disease compared to a subject that is homozygous for a PRNP allele encoding PrP but having a deletion that comprises 24 nucleotides within the nucleotide sequence encoding the octapeptide repeat region.

The present disclosure also provides methods of identifying a subject that is a candidate for treatment or prevention of a prion disease, the methods comprising: determining or having determined whether the subject comprises at least one reference PRNP allele or comprises at least one PRNP allele encoding an endogenous scrapie PrP, by: obtaining or having obtained a biological sample from the subject; and performing or having performed a sequence analysis on the biological sample to determine if the subject has a genotype comprising at least one reference PRNP allele or comprising at least one PRNP allele encoding an endogenous scrapie PrP; and wherein the presence of at least one reference PRNP allele or at least one PRNP allele encoding an endogenous scrapie PrP indicates the subject has an increased risk of developing a prion disease compared to a subject that is homozygous for a PRNP allele encoding PrP but having a deletion that comprises 24 nucleotides within the nucleotide sequence encoding the octapeptide repeat region, and is a candidate for treatment or prevention.

The present disclosure also provides nucleic acid molecules encoding a modified Prion protein PrP that comprises a nucleotide sequence encoding PrP but having a deletion that comprises 24 nucleotides within the nucleotide sequence encoding the octapeptide repeat region.

The present disclosure also provides antibodies to any PrP that does not have the deletion that comprises 24 nucleotides within the nucleotide sequence encoding the octapeptide repeat region.

The present disclosure also provides nucleic acid molecules encoding a modified PrP for use in treating a subject having a prion disease or at risk of developing a prion disease and comprising at least one reference PRNP allele or comprising at least one PRNP allele encoding an endogenous scrapie PrP, wherein the nucleic acid molecule encoding the modified PrP comprises a nucleotide sequence encoding PrP but having a deletion that comprises 24 nucleotides within the nucleotide sequence encoding the octapeptide repeat region.

### BRIEF DESCRIPTION OF THE DRAWINGS

FIG. 1 (Panels A and B) shows the nucleotide sequence (SEQ ID NO: 1) and amino acid sequence (SEQ ID NO: 2) of an octapeptide repeat region of PrP; Panel A shows deletion of a first region of the R3 to R4 region (underlined region; SEQ ID NO: 3); Panel B shows deletion of a second region of the R3 to R4 region (underlined region; SEQ ID NO: 4).

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horses, cows, sheep, and pigs), companion animals (such as, for example, dogs and cats), laboratory animals (such as, for example, mice, rats, and rabbits), and non-human primates. In some embodiments, the subject is a mammal. In some embodiments, the subject is a human. In some embodiments, the subject is a cow. In some embodiments, the subject is a sheep. In some embodiments, the human is a patient under the care of a physician.

It has been observed in accordance with the present disclosure that particular modifications of PrP may be protective against developing a prion disease. Thus, subjects that are carriers of at least one allele encoding such a modified PrP may be protected from developing a prion disease. Accordingly, subjects that are carriers of at least one allele encoding such a modified PrP may prevent or stop the initiation or progression of a prion disease.

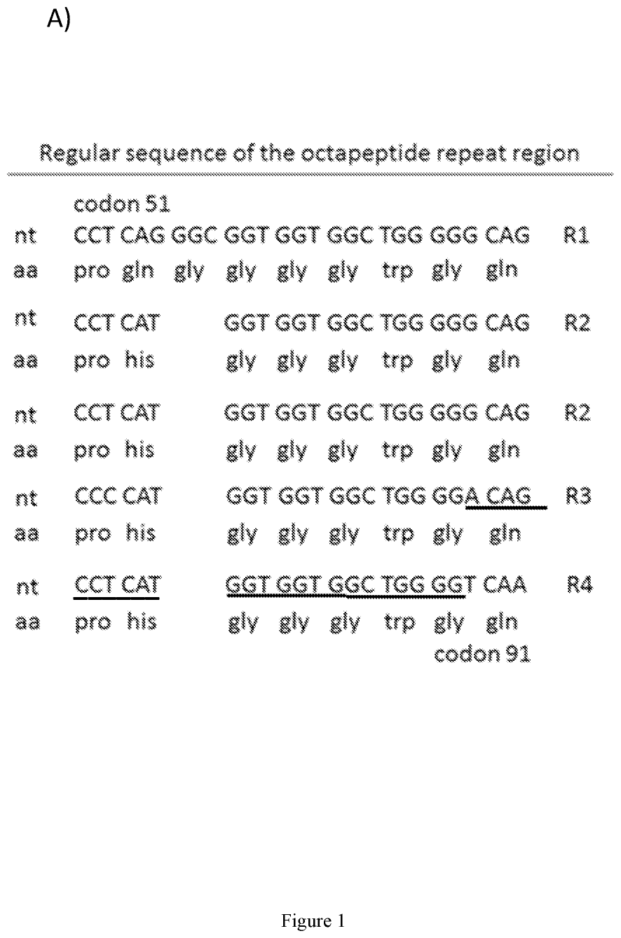

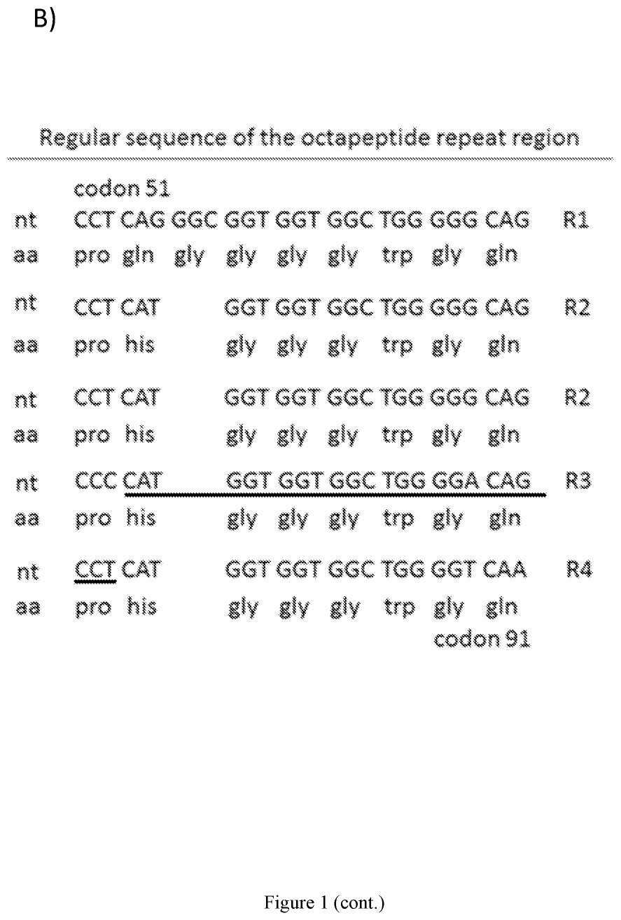

The octapeptide repeat region of reference PrP (i.e., the normal cellular isoform of PrP) is depicted in FIG. 1, Panels A and B (showing the nucleotide sequence (SEQ ID NO: 1) and the amino acid sequence (SEQ ID NO: 2)), and is comprised of regions R1, R2, R2, R3, and R4. The R2 region is repeated twice.

The present disclosure provides nucleic acid molecules encoding a modified PrP, wherein the nucleic acid molecules comprise a nucleotide sequence encoding PrP but having a deletion that comprises 24 nucleotides within the nucleotide sequence encoding the octapeptide repeat region. In some embodiments, the deletion comprises 24 nucleotides within the nucleotide sequence encoding the R2 to R4 region of the octapeptide repeat region. In some embodiments, the deletion comprises 24 nucleotides within the nucleotide sequence encoding the R3 to R4 region of the octapeptide repeat region. In some embodiments, the deletion comprises 24 nucleotides and comprises the deletion of ACAGCCT within the nucleotide sequence encoding the octapeptide repeat region. In some embodiments, the nucleic acid molecule encoding the modified PrP comprises a nucleotide sequence encoding PrP but having a deletion of ACAGCCTCATGGTGGTGGCTGGGG (SEQ ID NO: 3) (see underlined region of FIG. 1, Panel A) within the nucleotide sequence encoding the octapeptide repeat region of PrP. In some embodiments, the nucleic acid molecule encoding the modified PrP comprises a nucleotide sequence encoding PrP but having a deletion of CATGGTGGTGGCTGGGGACAGCCT (SEQ ID NO: 4) (see underlined region of FIG. 1, Panel B) within the nucleotide sequence encoding the octapeptide repeat region of PrP.

The present disclosure also provides modified PrP having a modified octapeptide repeat regions produced from nucleic acid molecules having the deletions disclosed herein.

The present disclosure provides methods of treating a subject having a prion disease or at risk of developing a prion disease. The methods comprise administering: a) a nucleic acid molecule encoding a modified PrP to the subject; or b) an inhibitory nucleic acid molecule that targets that targets a region of a PRNP that comprises a genetic variation that causes production of a scrapie PrP. In such methods, the subject comprises at least one reference PRNP allele or comprises at least one PRNP allele encoding an endogenous scrapie PrP. A “reference PRNP allele” is a PRNP allele that encodes a reference PrP (i.e., the normal cellular isoform of PrP which is properly folded and does not lead to a prion disease). A “PRNP allele encoding an endogenous scrapie PrP” is a PRNP allele that encodes a PrP that is misfolded (i.e., scrapie PrP) and may lead to a prion disease. The nucleic acid molecule encoding the modified PrP administered to the subject comprises a nucleotide sequence encoding PrP but having a deletion that comprises 24 nucleotides within the nucleotide sequence encoding the octapeptide repeat region.

In some embodiments, the methods comprise administering a nucleic acid molecule encoding a modified PrP to the subject. The nucleic acid molecule encoding the modified PrP comprises a nucleotide sequence encoding PrP but having a deletion that comprises 24 nucleotides within the nucleotide sequence encoding the octapeptide repeat region. In some embodiments, the 24 nucleotide deletion within the nucleotide sequence encoding the octapeptide repeat region is within the R2 to R4 region. In some embodiments, the 24 nucleotide deletion within the nucleotide sequence encoding the octapeptide repeat region is within the R3 to R4 region. In some embodiments, the deletion comprises 24 nucleotides and comprises the deletion of ACAGCCT within the nucleotide sequence encoding the octapeptide repeat region. In some embodiments, the nucleic acid molecule encoding the modified PrP comprises a nucleotide sequence encoding PrP but having a deletion of ACAGCCTCATGGTGGTGGCTGGGG (SEQ ID NO: 3) or having a deletion of CATGGTGGTGGCTGGGGACAGCCT (SEQ ID NO: 4) within the nucleotide sequence encoding the octapeptide repeat region of PrP. In some embodiments, the nucleic acid molecule encoding the modified PrP comprises a nucleotide sequence encoding PrP but having a deletion of ACAGCCTCATGGTGGTGGCTGGGG (SEQ ID NO: 3) within the nucleotide sequence encoding the octapeptide repeat region of PrP. In some embodiments, the nucleic acid molecule encoding the modified PrP comprises a nucleotide sequence encoding PrP but having a deletion of CATGGTGGTGGCTGGGGACAGCCT (SEQ ID NO: 4) within the nucleotide sequence encoding the octapeptide repeat region of PrP.

In some embodiments, the methods comprise administering an inhibitory nucleic acid molecule that targets that targets a region of a PRNP that comprises a genetic variation that causes production of a scrapie PrP. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target a region of a PRNP that comprises a genetic variation that causes production of a scrapie PrP. In some embodiments, the antisense RNA, siRNA, or shRNA hybridizes to a sequence within a PRNP genomic nucleic acid molecule or mRNA molecule that that comprises a genetic variation that causes production of a scrapie PrP and decreases expression of the PRNP polypeptide in a cell in the subject. In some embodiments, the inhibitory nucleic acid molecule comprises an antisense molecule that hybridizes to a PRNP genomic nucleic acid molecule or mRNA molecule that comprises a genetic variation that causes production of a scrapie PrP and decreases expression of the PRNP polypeptide in a cell in the subject. In some embodiments, the inhibitory nucleic acid molecule comprises an siRNA that hybridizes to a PRNP genomic nucleic acid molecule or mRNA molecule that comprises a genetic variation that causes production of a scrapie PrP and decreases expression of the PRNP polypeptide in a cell in the subject. In some embodiments, the inhibitory nucleic acid molecule comprises an shRNA that hybridizes to a PRNP genomic nucleic acid molecule or mRNA molecule that comprises a genetic variation that causes production of a scrapie PrP and decreases expression of the PRNP polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

In any of the embodiments described herein, the inhibitory nucleic acid molecules may be administered, for example, as one to two hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 mg to about 640 mg (2.5 to 9.14 mg/kg; 92.5 to 338 mg/m²—based on an assumption of a body weight of 70 kg and a conversion of mg/kg to mg/m² dose levels based on a mg/kg dose multiplier value of 37 for humans).

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In any of the methods disclosed herein, the subject comprises at least one reference PRNP allele that encodes a reference PrP (i.e., a PRNP allele that encodes the normal cellular isoform of PrP which is properly folded and does not lead to a prion disease) or comprises at least one PRNP allele encoding an endogenous scrapie PrP (i.e., a PRNP allele that encodes a misfolded PrP (i.e., scrapie PrP) and may lead to a prion disease). In some embodiments, the subject comprises a single reference PRNP allele. In some embodiments, the subject comprises two reference PRNP alleles. In some embodiments, the subject comprises a single PRNP allele encoding an endogenous scrapie PrP. In some embodiments, the subject comprises two PRNP alleles encoding an endogenous scrapie PrP. More broadly, a subjects' PRNP alleles can be categorized as homozygous protective PrP deletion on octapeptide repeat regions, heterozygous protective PrP deletion on octapeptide repeat regions, and protective PrP deletion null on octapeptide repeat regions. Subjects either null or heterozygous on protective PrP deletion on octapeptide repeat regions, regardless their endogenous scrapie PrP status, will be suitable for treatment as described herein.

In any of the methods disclosed herein, the endogenous scrapie PrP is any misfolded PrP. In some embodiments, the endogenous scrapie PrP comprises any one or more of the following polypeptide variations: P84S, P102L, P105L, P105S, P105T, G114V, A117V, G127V, M129V, G131V, S132I, A133V, Y145X, R148H, Q160X, Y163X, D167G, D167N, V176G, D188Efs25X, D178N-129V, D178N-129M, V180I, T183A, H187R, T188R, T188K, T188A, T193I, K194E, E196K, F198S, F198V, E200K, E200G, D202N, D202G, V203I, R208H, V210I, E211D, E211Q, Q212P, 1215V, Q217R, Y218N, E219K, A224V, Y226X, Q227X, M232R, P238S, 2-0PRD, 2-0PRI, 3-OPRI, 4-OPRI, 5-OPRI, 6-OPRI, 7-OPRI, 8-OPRI, 9-OPRI, and/or 12-OPRI. In some embodiments, the endogenous scrapie PrP comprises V180I, E200K, or V210I. In some embodiments, the endogenous scrapie PrP comprises E200K (rs28933385; 20:4699818:G:A).

In any of the methods disclosed herein, the nucleic acid molecule encoding the modified PrP may be administered to the subject by a variety of methods to achieve expression of the modified PrP. In some embodiments, the nucleic acid molecule encoding the modified PrP is administered to the subject by a vector to achieve episomal expression of the modified PrP. In some embodiments, the nucleic acid molecule encoding the modified PrP is administered to the subject by a vector to achieve episomal expression of the modified PrP. In some embodiments, the nucleic acid molecule encoding the modified PrP is administered to the subject by a viral vector to achieve episomal expression of the modified PrP. In some embodiments, the viral vector comprises adeno-associated virus (AAV) or a lentivirus. In some embodiments, the viral vector comprises AAV. In some embodiments, the viral vector comprises a lentivirus.

In some embodiments, the nucleic acid molecule encoding the modified PrP administered to the subject is inserted into the genome of the subject at the prion promoter site or a different site in the genome of the subject. In some embodiments, the nucleic acid molecule encoding the modified PrP administered to the subject is inserted into the genome of the subject using a clustered regularly interspaced short palindromic repeats and associated Cas9 endonuclease (CRISPR-Cas9)-based in vivo gene-editing therapy system. In some embodiments, the CRISPR-Cas9-based in vivo gene-editing therapy system comprises: the CRISPR-Cas9 system delivered in a vector; and a promotorless gene template delivered in a vector, wherein the promotorless gene template comprises the nucleic acid molecule encoding the modified PrP.

In some embodiments, the CRISPR-Cas9-based in vivo gene-editing therapy system induces one or more nicks or double-strand breaks at a recognition sequence(s) within a PRNP genomic nucleic acid molecule. The recognition sequence can be located within a coding region of the PRNP gene, or within regulatory regions that influence the expression of the gene. A recognition sequence can be located in an intron, an exon, a promoter, an enhancer, a regulatory region, or any non-protein coding region. The recognition sequence can include or be proximate to the start codon of the PRNP gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences.

Other nuclease agents and DNA-binding proteins that can also be used herein include, but are not limited to, zinc finger protein or zinc finger nuclease (ZFN) pair, Transcription Activator-Like Effector (TALE) protein, or Transcription Activator-Like Effector Nuclease (TALEN). The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a PRNP genomic nucleic acid molecule or it can be a nickase that creates a single-strand break in a PRNP genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of PRNP genomic nucleic acid molecules can be generated by contacting a cell with a Cas protein and one or more gRNAs that hybridize to one or more gRNA recognition sequences within a target genomic locus in the PRNP genomic nucleic acid molecule. The gRNA recognition sequence can include or be proximate to the start codon of a PRNP genomic nucleic acid molecule or the stop codon of a PRNP genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in a PRNP genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within a PRNP genomic nucleic acid molecule. An exemplary gRNA is a gRNA effective to direct a Cas enzyme to bind to or cleave a PRNP genomic nucleic acid molecule, wherein the gRNA comprises a DNA-targeting segment that hybridizes to a gRNA recognition sequence within the PRNP genomic nucleic acid molecule. Exemplary gRNAs comprise a DNA-targeting segment that hybridizes to a gRNA recognition sequence present within a PRNP genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the PRNP genomic nucleic acid molecule. The Cas protein can cleave the nucleic acid molecule at a site within or outside of the nucleic acid sequence present in the PRNP genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the PRNP genomic nucleic acid molecule to which a DNA-targeting segment of a gRNA will bind.

Such methods can result, for example, in a PRNP genomic nucleic acid molecule in which a region of the PRNP genomic nucleic acid molecule is disrupted, the start codon is disrupted, the stop codon is disrupted, or the coding sequence is disrupted or deleted. The cell can be further contacted with one or more additional gRNAs that hybridize to additional gRNA recognition sequences within the target genomic locus in the PRNP genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks, into which the promotorless gene template comprises the nucleic acid molecule encoding the modified PrP is inserted.

In some embodiments, the nucleic acid molecule encoding the modified PrP administered to the subject is inserted into the genome of the subject using a 5′-prime editing therapy.

The present disclosure also provides methods of treating a subject having a prion disease or at risk of developing a prion disease by administering a nucleic acid molecule encoding a modified PrP to the subject. The methods comprise: determining or having determined whether the subject comprises at least one reference PRNP allele that encodes a reference PrP (i.e., a PRNP allele that encodes the normal cellular isoform of PrP which is properly folded and does not lead to a prion disease) or comprises at least one PRNP allele encoding an endogenous scrapie PrP (i.e., a PRNP allele that encodes a misfolded PrP (i.e., scrapie PrP) and may lead to a prion disease), by: obtaining or having obtained a biological sample from the subject; and performing or having performed a sequence analysis on the biological sample to determine if the subject has a genotype comprising at least one reference PRNP allele or comprising at least one PRNP allele encoding an endogenous scrapie PrP. For those subjects that have at least one reference PRNP allele or have at least one PRNP allele encoding an endogenous scrapie PrP, the nucleic acid molecule encoding the modified PrP is administered or continued to be administered to the subject. The nucleic acid molecule encoding the modified PrP administered to the subject comprises a nucleotide sequence encoding PrP but having a deletion that comprises 24 nucleotides within the nucleotide sequence encoding the octapeptide repeat region. The presence of at least one reference PRNP allele or at least one PRNP allele encoding an endogenous scrapie PrP indicates the subject has an increased risk of developing a prion disease compared to a subject that is homozygous for a PRNP allele encoding PrP but having a deletion that comprises 24 nucleotides within the nucleotide sequence encoding the octapeptide repeat region.

Detecting the presence of at least one reference PRNP allele or at least one PRNP allele encoding an endogenous scrapie PrP in a biological sample from a subject can be carried out in vitro. In some embodiments, these methods can be carried out in situ. In some embodiments, these methods can be carried out in vivo. In any of these embodiments, the nucleic acid molecule can be present within a cell obtained from the subject. The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any PRNP nucleic acid molecule, preliminary processing designed to isolate or enrich the biological sample for the genomic DNA can be employed. A variety of techniques may be used for this purpose. When detecting the level of any PRNP nucleic acid molecule, different techniques can be used enrich the biological sample with mRNA molecules. Various methods to detect the presence or level of an mRNA molecule or the presence of a particular variant genomic DNA locus can be used.

In some embodiments, the methods described herein can be carried out by determining or having determined whether the subject comprises a misfolded PrP (i.e., a scrapie PrP), by: obtaining or having obtained a biological sample from the subject; and performing or having performed a sequence analysis on the biological sample to determine if the subject has a misfolded PrP (i.e., a scrapie PrP). For those subjects that have a misfolded PrP (i.e., a scrapie PrP), the nucleic acid molecule encoding the modified PrP is administered or continued to be administered to the subject. The nucleic acid molecule encoding the modified PrP administered to the subject comprises a nucleotide sequence encoding PrP but having a deletion that comprises 24 nucleotides within the nucleotide sequence encoding the octapeptide repeat region. The presence of a misfolded PrP (i.e., a scrapie PrP) indicates the subject has an increased risk of developing a prion disease compared to a subject that does not have a misfolded PrP (i.e., a scrapie PrP).

Detecting the presence a misfolded PrP (i.e., a scrapie PrP) in a biological sample from a subject can be carried out in vitro. In some embodiments, these methods can be carried out in situ. In some embodiments, these methods can be carried out in vivo. In any of these embodiments, the biological sample can be derived from cerebral spinal fluid. In some embodiments, scrapie PrP is determined from the cerebral spinal fluid of a subject.

In some embodiments, detecting at least one reference PRNP allele or at least one PRNP allele encoding an endogenous scrapie PrP in a subject comprises performing a sequence analysis on a biological sample obtained from the subject to determine the nucleotide sequence of a PRNP genomic nucleic acid molecule in the biological sample, and/or a PRNP mRNA molecule in the biological sample, and/or a PRNP cDNA molecule produced from an mRNA molecule in the biological sample. In some embodiments, the methods detect the PRNP genomic nucleic acid molecule that comprises one or more of the genetic variations described herein, or an mRNA molecule produced therefrom, or a cDNA molecule produced from the mRNA molecule, that result in a scrapie PrP.

In some embodiments, the methods of detecting the presence or absence of at least one reference PRNP allele or at least one PRNP allele encoding an endogenous scrapie PrP (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising a PRNP genomic nucleic acid molecule or mRNA molecule, and if mRNA, optionally reverse transcribing the mRNA into cDNA. Such assays can comprise, for example determining the identity of these positions of the particular PRNP nucleic acid molecule. In some embodiments, the method is an in vitro method.

In some embodiments, the assay comprises sequencing the entire nucleic acid molecule. In some embodiments, only a PRNP genomic nucleic acid molecule is analyzed. In some embodiments, only a PRNP mRNA is analyzed. In some embodiments, only a PRNP cDNA obtained from the PRNP mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the determining step, detecting step, or sequence analysis comprises: a) amplifying at least a portion of the PRNP nucleic acid molecule that encodes the PrP polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

In some embodiments, the probes (such as, for example, an alteration-specific probe) comprise a label. In some embodiments, the label is a fluorescent label, a radiolabel, or biotin.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The nucleic acid molecule encoding the modified PrP administered to the subject can be any of the nucleic acid molecules disclosed herein. For example, the nucleic acid molecule encoding the modified PrP administered to the subject may comprise a nucleotide sequence encoding PrP but having a deletion that comprises 24 nucleotides within the nucleotide sequence encoding the R2 to R4 region of the octapeptide repeat region. In some embodiments, the nucleic acid molecule encoding the modified PrP administered to the subject may comprise a nucleotide sequence encoding PrP but having a deletion that comprises 24 nucleotides within the nucleotide sequence encoding the R3 to R4 region of the octapeptide repeat region. In some embodiments, the nucleic acid molecule encoding the modified PrP administered to the subject may comprise a nucleotide sequence encoding PrP but having a deletion that comprises 24 nucleotides and comprises the deletion of ACAGCCT within the within the nucleotide sequence encoding the octapeptide repeat region. In some embodiments, the nucleic acid molecule encoding the modified PrP administered to the subject may comprises a nucleotide sequence encoding PrP but having a deletion of ACAGCCTCATGGTGGTGGCTGGGG (SEQ ID NO: 3) or having a deletion of CATGGTGGTGGCTGGGGACAGCCT (SEQ ID NO: 4) within the nucleotide sequence encoding the octapeptide repeat region of PrP.

The endogenous scrapie PrP can comprise any of the endogenous scrapie PrPs disclosed herein. For example, the endogenous scrapie PrP can comprise P84S, P102L, P105L, P105S, P105T, G114V, A117V, G127V, M129V, G131V, S132I, A133V, Y145X, R148H, Q160X, Y163X, D167G, D167N, V176G, D188Efs25X, D178N-129V, D178N-129M, V180I, T183A, H187R, T188R, T188K, T188A, T193I, K194E, E196K, F198S, F198V, E200K, E200G, D202N, D202G, V203I, R208H, V210I, E211D, E211Q, Q212P, 1215V, Q217R, Y218N, E219K, A224V, Y226X, Q227X, M232R, P238S, 2-0PRD, 2-0PRI, 3-OPRI, 4-OPRI, 5-OPRI, 6-OPRI, 7-OPRI, 8-OPRI, 9-OPRI, or 12-OPRI. In some embodiments, the endogenous scrapie PrP comprises V180I, E200K, or V210I. In some embodiments, the endogenous scrapie PrP comprises E200K.

In any of the methods described herein, the nucleic acid molecule encoding the modified PrP may administered to the subject by a vector to achieve episomal expression of the modified PrP, as described herein. In any of the methods described herein, the nucleic acid molecule encoding the modified PrP administered to the subject may be inserted into the genome of the subject at the prion promoter site or a different site in the genome of the subject, as described herein. In any of the methods described herein, the nucleic acid molecule encoding the modified PrP may administered to the subject may be inserted into the genome of the subject using a 5′-prime editing therapy, as described herein.

In any of the methods disclosed herein, the prion disease may be familial Creutzfeldt-Jakob disease (CJD), sporadic CJD, variant CJD, iatrogenic CJD, Gerstmann-Straussler-Scheinker (GSS) syndrome, fatal familial insomnia (FFI), prion protein amyloidosis, systemic amyloidosis, or prion protein cerebral amyloid angiopathy. In some embodiments, the prion disease is familial CJD. In some embodiments, the prion disease is sporadic CJD. In some embodiments, the prion disease is variant CJD. In some embodiments, the prion disease is iatrogenic CJD. In some embodiments, the prion disease is GSS syndrome. In some embodiments, the prion disease is FFI. In some embodiments, the prion disease is prion protein amyloidosis. In some embodiments, the prion disease is systemic amyloidosis. In some embodiments, the prion disease is prion protein cerebral amyloid angiopathy.

In any of the embodiments described herein, the subject in whom a prion disease is treated or prevented may be anyone at risk for developing a prion disease including, but not limited to, subjects with a genetic predisposition for developing a prion disease. Additional risk factors for a prion disease include, but are not limited to, subjects who are homozygous for the codon 129 polymorphism (Valine/Valine or Methionine/Methionine), subjects who have been exposed to scrapie PrP via a medical procedure, ingestion of meat or other tissues from an affected animal, or handling tissues or biofluids in a research setting. In any of the embodiments described herein, the methods can be used to improve a prion disease.

In any of the methods disclosed herein, the subject may be a mammal. In some embodiments, the mammal is a human. In some embodiments, the mammal is a cow or sheep. In some embodiments, the mammal is a cow. In some embodiments, the mammal is a sheep.

The present disclosure also provides methods of identifying a subject that is a candidate for treatment or prevention of a prion disease. The methods comprise: determining or having determined whether the subject comprises at least one reference PRNP allele that encodes a reference PrP (i.e., a PRNP allele that encodes the normal cellular isoform of PrP which is properly folded and does not lead to a prion disease) or comprises at least one PRNP allele encoding an endogenous scrapie PrP (i.e., a PRNP allele that encodes a misfolded PrP (i.e., scrapie PrP) and may lead to a prion disease), by: obtaining or having obtained a biological sample from the subject; and performing or having performed a sequence analysis on the biological sample to determine if the subject has a genotype comprising at least one reference PRNP allele or comprising at least one PRNP allele encoding an endogenous scrapie PrP. The presence of at least one reference PRNP allele or at least one PRNP allele encoding an endogenous scrapie PrP indicates the subject has an increased risk of developing a prion disease compared to a subject that is homozygous for a PRNP allele encoding PrP but having a deletion that comprises 24 nucleotides within the nucleotide sequence encoding the octapeptide repeat region, and is a candidate for treatment or prevention.

In any of the methods of identifying a candidate disclosed herein, the subject comprises at least one reference PRNP allele or comprises at least one PRNP allele encoding an endogenous scrapie PrP. In some embodiments, the subject comprises a single reference PRNP allele. In some embodiments, the subject comprises two reference PRNP alleles. In some embodiments, the subject comprises a single PRNP allele encoding an endogenous scrapie PrP. In some embodiments, the subject comprises two PRNP alleles encoding an endogenous scrapie PrP.

In any of the methods of identifying a candidate disclosed herein, the endogenous scrapie PrP comprises any one or more of the following polypeptide variations: P84S, P102L, P105L, P105S, P105T, G114V, A117V, G127V, M129V, G131V, S132I, A133V, Y145X, R148H, Q160X, Y163X, D167G, D167N, V176G, D188Efs25X, D178N-129V, D178N-129M, V180I, T183A, H187R, T188R, T188K, T188A, T193I, K194E, E196K, F198S, F198V, E200K, E200G, D202N, D202G, V203I, R208H, V210I, E211D, E211Q, Q212P, 1215V, Q217R, Y218N, E219K, A224V, Y226X, Q227X, M232R, P238S, 2-0PRD, 2-0PRI, 3-OPRI, 4-OPRI, 5-OPRI, 6-OPRI, 7-OPRI, 8-OPRI, 9-OPRI, and/or 12-OPRI. In some embodiments, the endogenous scrapie PrP comprises V180I, E200K, or V210I. In some embodiments, the endogenous scrapie PrP comprises E200K.

In any of the methods of identifying a candidate disclosed herein, the prion disease may be familial Creutzfeldt-Jakob disease (CJD), sporadic CJD, variant CJD, iatrogenic CJD, Gerstmann-Straussler-Scheinker (GSS) syndrome, fatal familial insomnia (FFI), prion protein amyloidosis, systemic amyloidosis, or prion protein cerebral amyloid angiopathy.

In any of the methods of identifying a candidate disclosed herein, the subject may be a mammal. In some embodiments, the mammal is a human. In some embodiments, the mammal is a cow or sheep.

In any of the methods of identifying a candidate disclosed herein, the methods can further comprises administering to a subject that is a candidate for treatment or prevention a nucleic acid molecule encoding a modified PrP that comprises a nucleotide sequence encoding PrP but having a deletion that comprises 24 nucleotides within the nucleotide sequence encoding the octapeptide repeat region. In some embodiments, the nucleic acid molecule encoding the modified PrP administered to the subject comprises a nucleotide sequence encoding PrP but having a deletion that comprises 24 nucleotides within the nucleotide sequence encoding the R2 to R4 region of the octapeptide repeat region. In some embodiments, the nucleic acid molecule encoding the modified PrP administered to the subject comprises a nucleotide sequence encoding PrP but having a deletion that comprises 24 nucleotides within the nucleotide sequence encoding the R3 to R4 region of the octapeptide repeat region. In some embodiments, the nucleic acid molecule encoding the modified PrP administered to the subject comprises a nucleotide sequence encoding PrP but having a deletion that comprises 24 nucleotides and comprises the deletion of ACAGCCT within the nucleotide sequence encoding the octapeptide repeat region. In some embodiments, the nucleic acid molecule encoding the modified PrP administered to the subject comprises a nucleotide sequence encoding PrP but having a deletion of ACAGCCTCA TGGTGGTGGCTGGGG (SEQ ID NO: 3) or having a deletion of CATGGTGGTGGCTGGGGACAGC CT (SEQ ID NO: 4) within the nucleotide sequence encoding the octapeptide repeat region of PrP.

In any of the methods of identifying a candidate described herein, the nucleic acid molecule encoding the modified PrP may administered to the subject by a vector to achieve episomal expression of the modified PrP, as described herein. In any of the methods described herein, the nucleic acid molecule encoding the modified PrP administered to the subject may be inserted into the genome of the subject at the prion promoter site or a different site in the genome of the subject, as described herein. In any of the methods described herein, the nucleic acid molecule encoding the modified PrP may administered to the subject may be inserted into the genome of the subject using a 5′-prime editing therapy, as described herein.

The present disclosure also provides nucleic acid molecules encoding a modified PrP for use in treating a subject having a prion disease or at risk of developing a prion disease and comprising at least one reference PRNP allele or comprising at least one PRNP allele encoding an endogenous scrapie PrP, wherein the nucleic acid molecule encoding the modified PrP comprises a nucleotide sequence encoding PrP but having a deletion that comprises 24 nucleotides within the nucleotide sequence encoding the octapeptide repeat region. Any of the nucleic acid molecules encoding a modified PrP disclosed herein can be used herein. The subject having a prion disease or at risk of developing a prion disease can comprise at least one PRNP allele encoding an endogenous scrapie PrP, wherein the endogenous scrapie PrP can be any of the endogenous scrapie PrPs described herein. The subject can have, or be at risk of developing, any of the prion diseases disclosed herein. The subject can be any of the mammals disclosed herein.

The present disclosure also provides nucleic acid molecules encoding a modified PrP for use in the preparation of a medicament for treating a subject having a prion disease or at risk of developing a prion disease and comprising at least one reference PRNP allele or comprising at least one PRNP allele encoding an endogenous scrapie PrP, wherein the nucleic acid molecule encoding the modified PrP comprises a nucleotide sequence encoding PrP but having a deletion that comprises 24 nucleotides within the nucleotide sequence encoding the octapeptide repeat region. Any of the nucleic acid molecules encoding a modified PrP disclosed herein can be used herein. The subject having a prion disease or at risk of developing a prion disease can comprise at least one PRNP allele encoding an endogenous scrapie PrP, wherein the endogenous scrapie PrP can be any of the endogenous scrapie PrPs described herein. The subject can have, or be at risk of developing, any of the prion diseases disclosed herein. The subject can be any of the mammals disclosed herein.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: PRNP Sequencing in Family

Sequencing was performed on a Libyan Jewish family composed of 22 members, 11 of which were asymptomatic carriers of the PRNP-E200K highly penetrant mutation (>94%). This family included a highly resilient and healthy 95 year old female that was well beyond the age at onset of 30-80 years, but never had Creutzfeldt-Jakob disease (CJD). Sequencing confirmed a deletion of 24 base pairs (8 amino acids), creating a new repeat sequence in the OPR (octapeptide repeat) of this individual that was validated by long read sequencing. This deletion, truncating part of R3 and R4 of the OPR, was on another allele different from the E200K variant this person carried.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having a prion disease or at risk of developing a prion disease, the method comprising:
a) administering a nucleic acid molecule encoding a modified Prion protein (PrP) to the subject; or
b) administering an inhibitory nucleic acid molecule that that targets a region of a prion nucleic acid molecule (PRNP) that comprises a genetic variation that causes production of a scrapie PrP to the subject;
wherein the subject comprises at least one reference PRNP allele or comprises at least one PRNP allele encoding an endogenous scrapie PrP;
wherein the nucleic acid molecule encoding the modified PrP administered to the subject comprises a nucleotide sequence encoding PrP but having a deletion that comprises 24 nucleotides within the nucleotide sequence encoding the octapeptide repeat region.

2. The method of claim 1, wherein the 24 nucleotide deletion within the nucleotide sequence encoding the octapeptide repeat region is within the R2 to R4 region.

3. The method of claim 1, wherein the 24 nucleotide deletion within the nucleotide sequence encoding the octapeptide repeat region is within the R3 to R4 region.

4. The method of claim 1, wherein the nucleic acid molecule encoding the modified PrP administered to the subject comprises a nucleotide sequence encoding PrP but having a deletion that comprises 24 nucleotides and comprises the deletion of ACAGCCT within the nucleotide sequence encoding the octapeptide repeat region.

5. The method of claim 1, wherein the nucleic acid molecule encoding the modified PrP administered to the subject comprises a nucleotide sequence encoding PrP but having a deletion of ACAGCCTCATGGTGGTGGCTGGGG (SEQ ID NO: 3) or having a deletion of CATGGTGGTGGCTGGGGACAGCCT (SEQ ID NO: 4) within the nucleotide sequence encoding the octapeptide repeat region of PrP.

6. The method of claim 1, wherein the endogenous scrapie PrP comprises P84S, P102L, P105L, P105S, P105T, G114V, A117V, G127V, M129V, G131V, S132I, A133V, Y145X, R148H, Q160X, Y163X, D167G, D167N, V176G, D188Efs25X, D178N-129V, D178N-129M, V180I, T183A, H187R, T188R, T188K, T188A, T193I, K194E, E196K, F198S, F198V, E200K, E200G, D202N, D202G, V203I, R208H, V210I, E211D, E211Q, Q212P, I215V, Q217R, Y218N, E219K, A224V, Y226X, Q227X, M232R, P238S, 2-0PRD, 2-0PRI, 3-OPRI, 4-OPRI, 5-OPRI, 6-OPRI, 7-OPRI, 8-OPRI, 9-OPRI, or 12-OPRI.

7. The method of claim 1, wherein the endogenous scrapie PrP comprises V180I, E200K, or V210I.

8. The method of claim 1, wherein the endogenous scrapie PrP comprises E200K.

9. The method of claim 1, wherein the nucleic acid molecule encoding the modified PrP is administered to the subject by a vector to achieve episomal expression of the modified PrP.

10-11. (canceled)

12. The method of claim 1, wherein the nucleic acid molecule encoding the modified PrP administered to the subject is inserted into the genome of the subject at the prion promoter site or a different site in the genome of the subject.

13-15. (canceled)

16. The method of claim 1, wherein the prion disease is familial Creutzfeldt-Jakob disease (CJD), sporadic CJD, variant CJD, iatrogenic CJD, Gerstmann-Straussler-Scheinker (GSS) syndrome, fatal familial insomnia (FFI), prion protein amyloidosis, systemic amyloidosis, or prion protein cerebral amyloid angiopathy.

17-19. (canceled)

20. A method of treating a subject having a prion disease or at risk of developing a prion disease by administering a nucleic acid molecule encoding a modified Prion protein (PrP) to the subject, the method comprising:
determining or having determined whether the subject comprises at least one reference PRNP allele or comprises at least one PRNP allele encoding an endogenous scrapie PrP, by:
obtaining or having obtained a biological sample from the subject; and
performing or having performed a sequence analysis on the biological sample to determine if the subject has a genotype comprising at least one reference PRNP allele or comprising at least one PRNP allele encoding an endogenous scrapie PrP; and
administering or continuing to administer the nucleic acid molecule encoding the modified PrP to the subject that has at least one reference PRNP allele or has at least one PRNP allele encoding an endogenous scrapie PrP;
wherein the nucleic acid molecule encoding the modified PrP administered to the subject comprises a nucleotide sequence encoding PrP but having a deletion that comprises 24 nucleotides within the nucleotide sequence encoding the octapeptide repeat region; and
wherein the presence of at least one reference PRNP allele or at least one PRNP allele encoding an endogenous scrapie PrP indicates the subject has an increased risk of developing a prion disease compared to a subject that is homozygous for a PRNP allele encoding PrP but having a deletion that comprises 24 nucleotides within the nucleotide sequence encoding the octapeptide repeat region.

21. The method of claim 20, wherein the nucleic acid molecule encoding the modified PrP administered to the subject comprises a nucleotide sequence encoding PrP but having a deletion that comprises 24 nucleotides within the nucleotide sequence encoding the R2 to R4 region of the octapeptide repeat region.

22. The method of claim 20, wherein the nucleic acid molecule encoding the modified PrP administered to the subject comprises a nucleotide sequence encoding PrP but having a deletion that comprises 24 nucleotides within the nucleotide sequence encoding the R3 to R4 region of the octapeptide repeat region.

23. The method of claim 20, wherein the nucleic acid molecule encoding the modified PrP administered to the subject comprises a nucleotide sequence encoding PrP but having a deletion that comprises 24 nucleotides and comprises the deletion of ACAGCCT within the nucleotide sequence encoding the octapeptide repeat region.

24. The method of claim 20, wherein the nucleic acid molecule encoding the modified PrP administered to the subject comprises a nucleotide sequence encoding PrP but having a deletion of ACAGCCTCATGGTGGTGGCTGGGG (SEQ ID NO: 3) or having a deletion of CATGGTGGTGGCTGGGGACAGCCT (SEQ ID NO: 4) within the nucleotide sequence encoding the octapeptide repeat region of PrP.

25. The method of n m claim 20, wherein the endogenous scrapie PrP comprises P84S, P102L, P105L, P105S, P105T, G114V, A117V, G127V, M129V, G131V, S132I, A133V, Y145X, R148H, Q160X, Y163X, D167G, D167N, V176G, D188Efs25X, D178N-129V, D178N-129M, V180I, T183A, H187R, T188R, T188K, T188A, T193I, K194E, E196K, F198S, F198V, E200K, E200G, D202N, D202G, V203I, R208H, V210I, E211D, E211Q, Q212P, I215V, Q217R, Y218N, E219K, A224V, Y226X, Q227X, M232R, P238S, 2-0PRD, 2-OPRI, 3-OPRI, 4-OPRI, 5-OPRI, 6-OPRI, 7-OPRI, 8-OPRI, 9-OPRI, or 12-OPRI.

26. The method of claim 20, wherein the endogenous scrapie PrP comprises V180I, E200K, or V210I.

27. The method of claim 20, wherein the endogenous scrapie PrP comprises E200K.

28. The method of claim 20, wherein the nucleic acid molecule encoding the modified PrP is administered to the subject by a vector to achieve episomal expression of the modified PrP.

29-30. (canceled)

31. The method of claim 20, wherein the nucleic acid molecule encoding the modified PrP administered to the subject is inserted into the genome of the subject at the prion promoter site or a different site in the genome of the subject.

32-34. (canceled)

35. The method of claim 20, wherein the prion disease is familial Creutzfeldt-Jakob disease (CJD), sporadic CJD, variant CJD, iatrogenic CJD, Gerstmann-Straussler-Scheinker (GSS) syndrome, fatal familial insomnia (FFI), prion protein amyloidosis, systemic amyloidosis, or prion protein cerebral amyloid angiopathy.

36-38. (canceled)

39. A method of identifying a subject that is a candidate for treatment or prevention of a prion disease, the method comprising:
determining or having determined whether the subject comprises at least one reference PRNP allele or comprises at least one PRNP allele encoding an endogenous scrapie PrP, by:
obtaining or having obtained a biological sample from the subject; and
performing or having performed a sequence analysis on the biological sample to determine if the subject has a genotype comprising at least one reference PRNP allele or comprising at least one PRNP allele encoding an endogenous scrapie PrP; and
wherein the presence of at least one reference PRNP allele or at least one PRNP allele encoding an endogenous scrapie PrP indicates the subject has an increased risk of developing a prion disease compared to a subject that is homozygous for a PRNP allele encoding PrP but having a deletion that comprises 24 nucleotides within the nucleotide sequence encoding the octapeptide repeat region, and is a candidate for treatment or prevention.

40. The method of claim 39, wherein the endogenous scrapie PrP comprises P84S, P102L, P105L, P105S, P105T, G114V, A117V, G127V, M129V, G131V, S132I, A133V, Y145X, R148H, Q160X, Y163X, D167G, D167N, V176G, D188Efs25X, D178N-129V, D178N-129M, V180I, T183A, H187R, T188R, T188K, T188A, T193I, K194E, E196K, F198S, F198V, E200K, E200G, D202N, D202G, V203I, R208H, V210I, E211D, E211Q, Q212P, I215V, Q217R, Y218N, E219K, A224V, Y226X, Q227X, M232R, P238S, 2-0PRD, 2-0PRI, 3-OPRI, 4-OPRI, 5-OPRI, 6-OPRI, 7-OPRI, 8-OPRI, 9-OPRI, or 12-OPRI.

41. The method of claim 39, wherein the endogenous scrapie PrP comprises V180I, E200K, or V210I.

42. The method of claim 39, wherein the endogenous scrapie PrP comprises E200K.

43. The method of claim 39, wherein the prion disease is familial Creutzfeldt-Jakob disease (CJD), sporadic CJD, variant CJD, iatrogenic CJD, Gerstmann-Straussler-Scheinker (GSS) syndrome, fatal familial insomnia (FFI), prion protein amyloidosis, systemic amyloidosis, or prion protein cerebral amyloid angiopathy.

44-46. (canceled)

47. The method of claim 39, the method further comprising administering to a subject that is a candidate for treatment or prevention a nucleic acid molecule encoding a modified PrP that comprises a nucleotide sequence encoding PrP but having a deletion that comprises 24 nucleotides within the nucleotide sequence encoding the octapeptide repeat region.

48-58. (canceled)

59. A nucleic acid molecule encoding a modified Prion protein (PrP) that comprises a nucleotide sequence encoding PrP but having a deletion that comprises 24 nucleotides within the nucleotide sequence encoding the octapeptide repeat region.

60-75. (canceled)


SEQUENCE LISTING (4 sequences)

SEQ ID NO: 1 - DNA, 123 bp, synthetic construct
  source 1..123: mol_type = other DNA
cctcagggcggtggtggctgggggcagcctcatggtggtggctgggggcagcctcatggtggtggctgggggcagccccatggtggtggctggggacagcctcatggtggtggctggggtcaa

SEQ ID NO: 2 - protein, 41 aa, synthetic construct
PQGGGGWGQPHGGGWGQPHGGGWGQPHGGGWGQPHGGGWGQ

SEQ ID NO: 3 - DNA, 24 bp, synthetic construct
  source 1..24: mol_type = other DNA
acagcctcatggtggtggctgggg

SEQ ID NO: 4 - DNA, 24 bp, synthetic construct
  source 1..24: mol_type = other DNA
catggtggtggctggggacagcct